(19) United States
(12) Patent Application Publication
(10) Pub. No.: US 2012/0100155 A1
(43) Pub. Date: Apr. 26, 2012

(54) PHARMACEUTICAL AGENT

(75) Inventors: Gregory Stoloff, London (GB); Wilson Wanderley, Aylesbury Buckinghamshire (GB)

(73) Assignee: PEPTCELL LIMITED, London (GB)

(21) Appl. No.: 13/379,996

(22) PCT Filed: Jun. 22, 2010
(86) PCT No.: PCT/EP10/58788

§ 371 Date: Dec. 21, 2011

(30) Foreign Application Priority Data
Jun. 22, 2009 (GB) 0910754.1
Aug. 10, 2009 (GB) 0913956.9
Nov. 27, 2009 (GB) 0920790.3

(51) Int. Cl. A61K 39/395; C12N 15/63; C07K 14/715; A61P 11/06; A61K 31/7088; A61P 29/00; A61P 11/00; C07K 16/24; A61K 38/02

(52) U.S. Cl. 424/158.1; 530/389.2; 435/320.1; 530/350; 514/1.1; 514/44 R

(57) ABSTRACT

Provided is a pharmaceutical agent for use in the treatment of inflammation, in a subject prone to and/or experiencing an excessive inflammatory response as a result of infection with an infectious agent and/or exposure to an allergen and/or exposure to an environmental trigger, which pharmaceutical agent comprises an agent for preventing, hindering, modulating or reducing: (a) the production, activity and/or effect of one or more cytokines; and/or (b) the functionality of one or more cells that are targets for the cytokines; and/or (c) a pathological effect caused by cells producing and/or activated by the cytokines.

The present invention concerns a pharmaceutical agent for the treatment of inflammation, particularly inflammation of lung tissue, caused by infectious agents (such as influenza), by allergens, and/or by environmental triggers. The invention also concerns corresponding pharmaceutical compositions. The invention relates especially to the treatment of patients suffering from excessive inflammatory response of any cause, but in particular to treatment of those suffering from infection with annual/pandemic influenza where the disease progression is causing effects that could be life threatening. Such patients are prone to an unbalanced immune response leading to serious and sometimes life-threatening symptoms.

It has been known for many years that the influenza virus is constantly mutating. As a result, a new influenza virus strain appears during each annual influenza season, requiring development of a new influenza vaccine for each season. The influenza vaccine, like all vaccines, promotes development of immune system defenses, in this case a neutralising antibody response, to the influenza virus in order that the subject is able to fight the infection before it causes illness.

Despite the requirement for a vaccine for each new virus strain, most individuals contracting annual flu who have not been vaccinated will nonetheless have some degree of immune protection against the new virus. This is because the mutations that give rise to the new virus are relatively small, and hence an individual's pre-existing antibody response is still able to provide some degree of protection against the new virus.

More recently, this pre-existing antibody response has been found to play a significant role in reducing the likelihood of a subject becoming seriously ill or dying as a result of contracting influenza. When an individual's pre-existing antibody response has very little or no capacity to neutralise the new influenza virus strain, the natural cellular immune response that the individual will develop to this new strain will become dominant over the antibody response and develop into an uncontrolled inflammatory response leading to severe lung pathology and even death. This is due to the role played by antibodies in modulating the cellular, and its associated cytokine, immune response. Cytokines are produced by many different cell types, some immune and some non-immune cells, and they determine the type and the proliferation rate of immune cells engaged in fighting the viral infection. In the absence of a neutralising antibody response the type and level of the cellular immune response, and the cytokine environment created as a result, both change and are significantly increased. This increased cellular and cytokine response in the infected lungs, which is characteristically defined as inflammatory, is entirely capable of clearing the virus, but can cause the individual to develop severe impairment of lung function (e.g. oedema, bronchoconstriction, etc.) leading to death in the most severe cases.

It has been known for many years that several cytokines are involved in causing this problem. TNF-α, IL-12 and IFN-γ are three of the most significant cytokines operating in this respect. The cytokine IFN-γ has received particular attention. Baumgarth and Kelso (Journal of Virology, 1996, 70(7):4411-4418) had recognised that neutralisation of IFN-γ could lead to a significant reduction in the magnitude of the cellular infiltrate in lung tissue following infection. In three independent experiments mice were treated intravenously either with anti-IFN-γ monoclonal antibodies, or with rat immunoglobulin, before being infected with influenza virus. However, it was found that although mice from both groups developed signs of clinical illness, they recovered'from infection at similar rates.

From this study, and similar studies, the inventors considered whether the suppression of IFN-γ, TNF-α, IL-12, and other cytokines might be a useful treatment for inflammation arising out of influenza, and other pathogens. Pandemic influenza, by its nature, is a particular type of flu virus that has not been seen by the population before. Rather than appearing from a mutation in a prior virus strain (antigenic drift), as is the case in annual influenza, pandemic influenza appears as a result of a significant change in the genetic makeup (antigenic shift) of an influenza virus, giving rise to a new influenza strain that has never circulated before amongst humans. This may happen when a flu virus jumps species, such as avian flu or swine flu moving into the human population. Alternatively, the new pandemic strain may arise as a result of the avian or swine influenza exchanging genetic material with the human influenza through a process called genetic reassortment. Although referred to in here as a pandemic influenza for ease of definition, this new pandemic strain will only cause a pandemic if the virus can spread easily amongst humans whilst causing serious illness.

It is important to emphasize that because the pandemic virus is a completely novel influenza strain that has not circulated before in the population, humans will not possess any pre-existing antibody protection against it. Accordingly, the pathological problems described earlier, which are associated with an increased cellular response in the infected lungs, will be more prevalent and/or significant during a pandemic outbreak of influenza. This would be particularly relevant amongst healthier individuals (e.g. young adults) whose strong and responsive immune systems may more easily overreact and cause particularly acute symptoms. Such symptoms may also be found in responses to other pathogens, allergens and environmental triggers.

Because of the central role played by inflammatory cytokines, it is an aim of the present invention to develop a treatment for modulating the cytokine profile that arises at a very specific time in the progression of an influenza infection, or that arises out of the progression of infection with other pathogens or exposure to allergens and environmental triggers. This treatment will result in a reduction in the proliferation and degree of response of specific immune cell populations in the tissue involved (e.g. lung tissue), which in turn will minimise the dangerous clinical complications associated with infection by pandemic viruses, or associated with exposure to allergens and environmental triggers. Surprisingly, the inventors have found that the use of agents for modulating, preventing, hindering or reducing the production of certain cytokines (such as anti-cytokine antibodies, including soluble cytokine receptors, and including anti-TNF-α, anti-IL-12 and anti-IFN-γ antibodies) at specific times post-infection or post exposure, can significantly improve survival rates and morbidity scores.

Accordingly, the present invention provides pharmaceutical agent for use in the treatment of inflammation, in a subject prone to and/or experiencing an excessive inflammatory response as a result of infection with an infectious agent and/or exposure to an allergen and/or exposure to an environmental trigger, which pharmaceutical agent comprises an agent for preventing, hindering, modulating or reducing: (a) the production, activity and/or effect of one or more cytokines; and/or (b) the functionality of one or more cells that are targets for the cytokines; and/or (c) a pathological effect caused by cells producing and/or activated by the cytokines.

Typically in the present invention, the cytokine is an inflammatory cytokine (or a proinflammatory cytokine) and the inflammatory response is an inflammatory (or proinflammatory) cytokine response.

Numerous studies have been conducted looking at modulating the cytokine response in the initial phase (i.e. innate phase) of the immune response to the virus, bacterium, fungus, allergen or environmental trigger following infection or exposure. However, none has shown any significant advantage over treatment with placebo. The inventors have recognised these findings. However, the inventors have determined that modulation of the cytokine response in the middle stage of the immune response (i.e. early stage of the adaptive response) to the pathogen, which in the case of the influenza virus typically starts 48 to 96 hours post-infection (or 48 to 96 hours after onset of symptoms in humans) results in a significant reduction in the mortality and morbidity of infected or affected animals compared to animals treated with placebo.

The present invention will now be described in more detail by way of example only with reference to the following Figures.

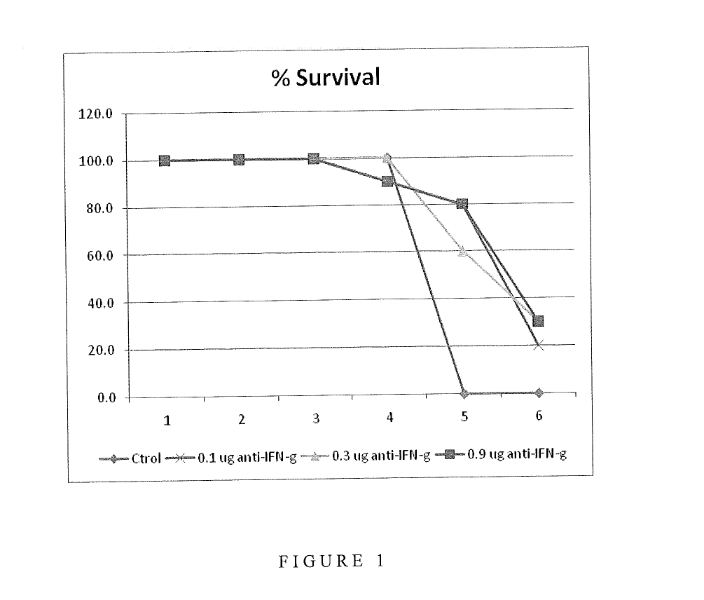

FIG. 1 shows the survival rate of mice challenged with influenza and treated with the agent of the invention two days after challenging with influenza.

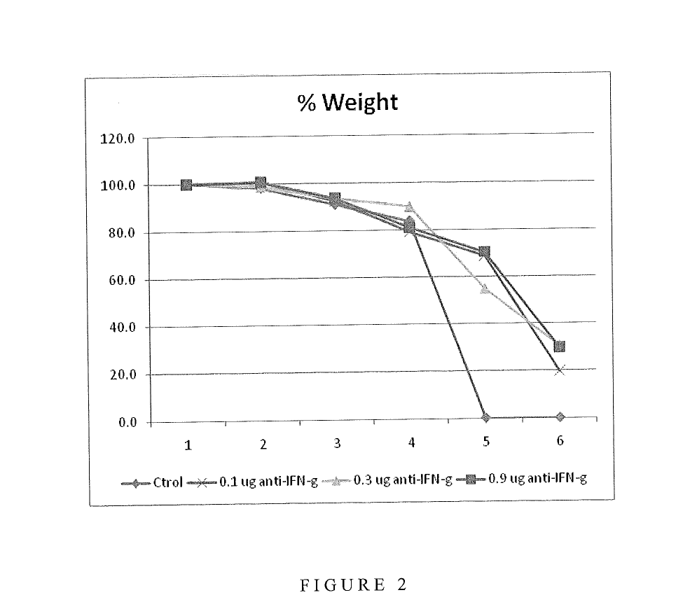

FIG. 2 shows the day-by-day change in weight of the mice in each group (as the percentage of starting weight averaged over the surviving mice). Note: mice were culled when reaching determined weight loss and morbidity milestones in accordance with the Scientific Procedures Act 1986.

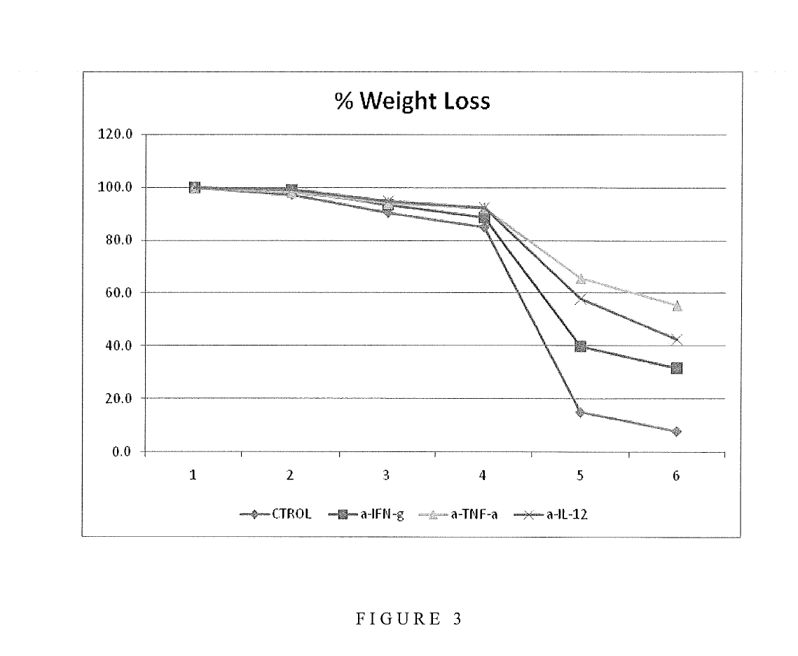

FIG. 3 shows % weight loss for four groups (A control, B anti-IFN-γ, C anti-TNF-α, and D anti-IL-12). Weight loss is represented as the average percentage weight of animals within a group on a daily basis compared to the average weight of the same group at the start of the study (day 1).

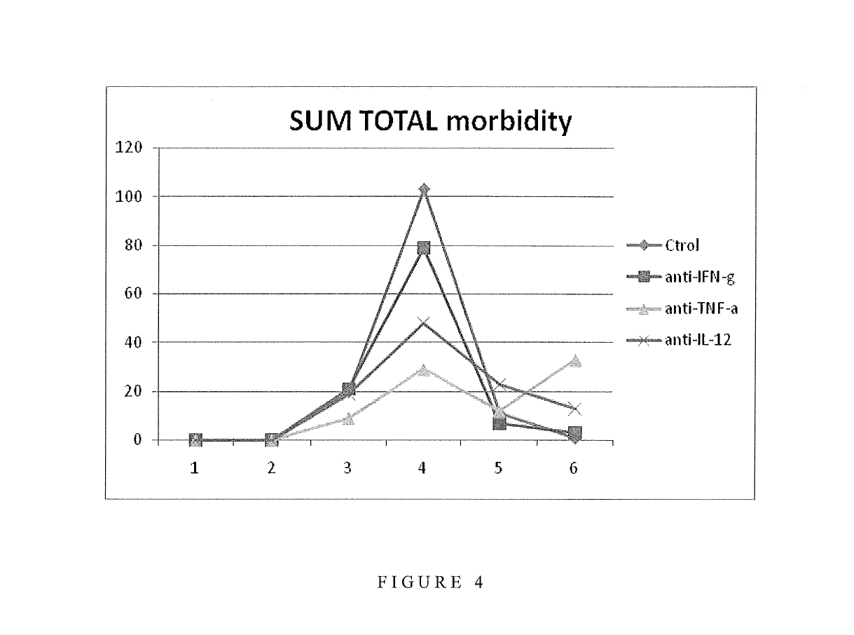

FIG. 4 shows sum total morbidity for four groups (A control, B anti-IFN-γ, C anti-TNF-α, and D anti-IL-12). Sum total morbidity is represented as the sum of all the morbidity scores for all animals still alive within a group at the end of each day.

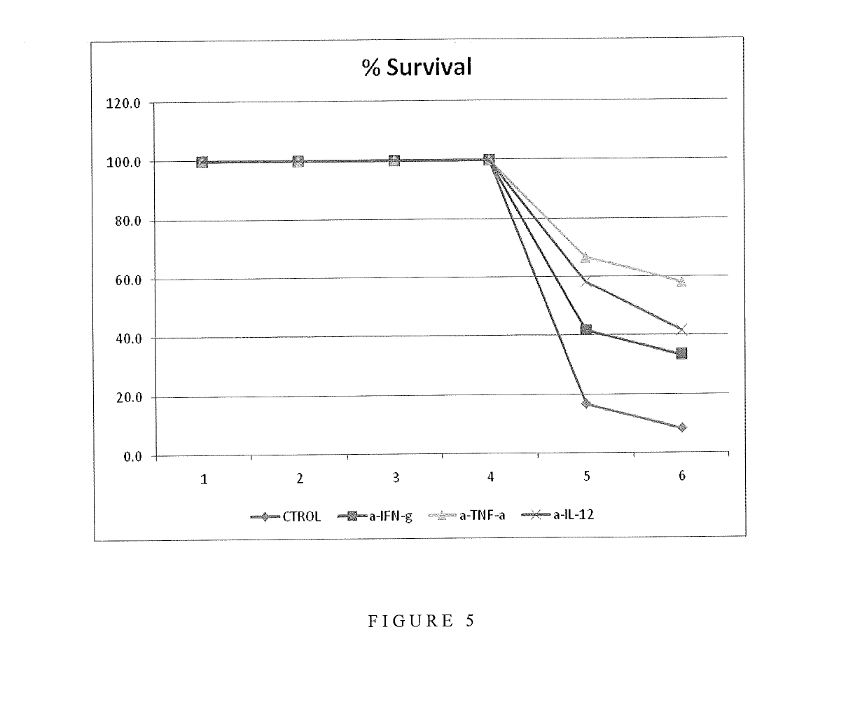

FIG. 5 shows % survival for four groups (A control, B anti-IFN-γ, C anti-TNF-α, and D anti-IL-12). Survival is represented as the percentage animals within a group alive at the end of each day compared to the number of animals that started the study (day 1).

As has been mentioned, the present invention is concerned with subjects prone to and/or experiencing an excessive inflammatory response as a result of infection with an infectious agent and/or exposure to an allergen and/or exposure to an environmental trigger. The type of inflammation will depend upon the infectious agent or the allergen/trigger, and may be acute, subacute or chronic. Preferably the inflammation is acute (such as eosinophilic) inflammation, or is chronic (such as Delayed Type Hypersensitivity—DTH) inflammation.

The infectious agents, allergens and environmental triggers are not especially limited, and may be any such agents allergens or triggers that cause an excessive inflammatory response. Thus, the infectious agent may be any pathogen capable of inducing acute and/or chronic inflammation. Similarly, the allergen or environmental trigger (including both known and unknown environmental triggers) may be any such allergen or trigger capable of inducing acute and/or chronic inflammation. In the present context, an allergen may be any substance which causes an excessive inflammatory response in a subject exposed to it. Similarly, an environmental trigger may be any environmental condition or environmental substance which causes an excessive inflammatory response in a subject exposed to it (conditions may include anything that is not a substance, such as temperature, humidity, sunlight etc., whilst environmental substances may include any substances which are not normally considered to be allergens, such as poisons, chemical waste, radioactive waste and the like). In more preferred embodiments, the infectious agent is a virus, a bacterium or a fungus. More preferably the infectious agent may be any one or more of the following: an influenza virus, haemophilus influenza, SARS virus, adenovirus, respiratory syncitial virus, streptococcus spp, staphylococcus spp, legionella spp, pseudomonas spp, klebsiella spp, burkholderia spp, pneumococcus spp, Mycobacterium spp, Chlamydia spp, blastomyces spp, cryptococcus spp, and aspergillus spp.

Preferably the allergen is an allergen that may cause Asthma, and the environmental trigger is a trigger that may cause Chronic Obstructive Pulmonary Disease (COPD). In more preferred embodiments, the infectious agent, allergen or trigger are capable of inducing a pathogenic pro-inflammatory response in the respiratory tract.

As has been indicated, the agent may operate by preventing, hindering, modulating or reducing any or all of the following: (a) the production, activity and/or effect of one or more cytokines; and/or (b) the functionality of one or more cells that are targets for the cytokines; and/or (c) a pathological effect caused by cells producing and/or activated by the cytokines. Provided that the ability of the agent to take effect in these ways is not hindered, the nature of the agent is not especially limited.

In the context of the present invention, an inflammatory cytokine (or a proinflammatory cytokine) is a cytokine which promotes inflammation. The present invention extends to agents affecting production or activity of any inflammatory cytokine (either directly or indirectly), including any interleukin, any chemokine, any TNF and any interferon. Thus, the cytokines involved may be any one or more of the following:

| | |
|---|---|
| Interleukins | |
| IL-1 superfamily: | 1 (1Ra) 18 33 |
| IL-6 like/gp 130-using: | 6, 11, 27, 30, 31 (+non IL Oncostatin M, |
| | Leukemia inhibitory factor, Ciliary |
| | neurotrophic factor, Cardiotrophin 1) |
| Interferon type III: | 28, 29 |
| Common γ-chain | 2/15, 3, 4, 7, 9, 13, 21 |
| family: | |
| IL-12 family: | 12, 23, 27, 35 |
| Other: | 5, 8, 14, 16, 17/25, (A), 32 |
| Chemokines | |
| CCL | 1, 2, 3, 4, 5, 6, 7, 8, 9, 10, 11, 12, 13, 14, 15, |
| | 16, 17, 18, 19, 20, 21, 22, 23, 24, 25, 26, 27, 28 |
| CXCL | 1, 2, 3, 4, 5, 6, 7, 8, 9, 10, 11, 12, 13, 14, 15, |
| | 16, 17 |
| CX3CL | 1 |
| XCL | 1, 2 |
| TNFs | |
| Main | TNF-alpha |
| TNF (ligand) | 4-1BB ligand, B-cell activating factor, |
| superfamily | FAS ligand |
| Lymphotoxin | OX40L, RANKL, TRAIL |
| Cluster of | CD70, CD153, CD154 |
| differentiation | |
| Interferons | |
| I | alpha (Pegylated 2a, Pegylated 2b), |
| | beta (1a, 1b) |
| II | gamma |
| III | 28, 29 |
| Other | |

Monokine Lymphokine (Lymphotoxin, Transfer factor) Growth
factor Hematopoietic (Stem cell factor, Colony-stimulating
factor) Autocrine motility factor Osteopontin.

In particularly preferred embodiments of the invention the cytokines involved are IFN-γ, TNF-α, and/or IL-12. It is particularly preferred that the cytokine is IFN-γ.

As has been stated, provided that the ability of the agent to take effect in the ways described above is not hindered, the nature of the agent is not especially limited. It is particularly preferred, however, that the agent is an anti-cytokine (an anti-proinflammatory cytokine) antibody or a soluble form of the cytokine receptor. The agent may be targeted to any of the cytokines referred to above. Thus, when it is an anti-cytokine antibody it may be an antibody against any of the cytokines referred to above, and when it is a soluble form of a receptor it may be a soluble form of a receptor for any of the cytokines referred to above. However, preferably it is an anti-IFN-γ antibody, an anti-TNF-α antibody, or an anti-IL-12 antibody, or alternatively a soluble form of: an IFN-γ receptor, a TNF-α receptor, or an IL-12 receptor.

The anti-cytokine antibody or soluble cytokine receptor may have the effect (either directly or indirectly) of preventing, hindering, modulating or reducing any or all of: (a) the production, activity and/or effect of one or more cytokines; and/or (b) the functionality of one or more cells that are targets for the cytokines; and/or (c) a pathological effect caused by cells producing and/or activated by the cytokines. However, typically the anti-cytokine antibody or the soluble receptor will have the direct effect of preventing, hindering, modulating or reducing the production or activity of inflammatory (proinflammatory) cytokines. In the case of the anti-cytokine antibody, this will be mediated by the antibody binding directly to the cytokine and preventing it from having its usual effect, although it is not excluded that the antibody may induce apoptosis of immune cells interacting directly with the targeted cytokine. Similarly the soluble cytokine receptor will bind and neutralise circulating cytokines before they reach their natural target cells and prevent the cytokine from having its usual effect. Neither of these activities is necessarily superior or more effective than the other and hence they will all have the effect of preventing, hindering, modulating or reducing (a) a pathological effect caused by cells producing or activated by the inflammatory cytokine and/or (b) a functionality of the cells that are target of the proinflammatory cytokine. Often, the binding of cytokine to a cell causes production of yet more cytokine in a cascade, and this preventing such binding may also cause prevention of cytokine production.

The anti-cytokine antibody used in the present invention is not especially limited and may be any anti-cytokine antibody provided that it has one or more of the effects required. Thus the antibody may be a monoclonal or polyclonal antibody, or may be an antibody fragment that retains the required effect. Preferably the antibody is a licensed and commercially available antibody such as Remicade® (infliximab), Humira® (adalimumab), Cimzia® (certolizumab) or Simponi® (golimumab).

The cytokine receptor used in the present invention is not especially limited and may be any cytokine receptor provided that it is soluble and has one or more of the effects required. Thus the receptor may be a modified (solubilised) receptor, or may be a fragment of a receptor, either free or conjugated to another product or compound, that retains the required effect. These soluble receptors typically retain the functionality (i.e. cytokine binding capacity) of the naturally occurring membrane bound forms of the receptor, but typically lack the transmembrane and/or signal transduction domains of the membrane bound molecule. They may be produced by standard known recombinant means. Preferably the soluble receptor is a licensed and commercially available product such as Enbrel® (etanercept).

The subject of an excessive inflammatory response to the infection is not especially limited and may be any subject prone to such a response. Similarly, the type of tissue in the subject that is displaying the inflammatory response is not especially limited, and will depend on the infectious agent, allergen or environmental agent that the subject has been exposed to. Preferably the tissue comprises lung, liver, intestinal epithelia and any tissues whose metabolic function is altered and/or viability is reduced by the action of an inflammatory response triggered by infection, allergen or environmental factor. In the most preferred embodiments, the tissue is lung tissue. For example, subjects suffering from influenza and/or asthma may typically be those in which lung tissue is involved. Typical subjects falling under this category of patient will be described in more detail below.

In a pandemic influenza situation the subject is typically a person who is regarded as being a healthy individual. Generally the subject in a pandemic setting is not ‘old’ or ‘young’ (for example, an individual from 13-65 years old, and preferably (more commonly) 20-50 years old).

Alternatively, and independently of whether or not the influenza is a pandemic influenza, the subject is a subject in whom symptoms of respiratory difficulty arise and/or in whom cytokine levels (any of the above mentioned cytokines, but typically IFN-γ, TNF-α and/or IL-12) increase at the onset of symptoms of respiratory difficulty. More preferably, the subject is a subject in whom symptoms of respiratory difficulty arise, and/or in whom cytokine levels increase, at the following times after onset of influenza symptoms: from 36 hours or more (more preferably from 48 hours or more, from 60 hours or more, or from 72 hours or more; most preferably from 36-96 hours, from 48-96 hours, from 60-96 hours or from 72-96 hours). Alternatively, and independently of whether or not the pathogen is a pandemic influenza, the subject is a subject in whom symptoms of respiratory difficulty arise and/or in whom cytokine levels increase, at the onset (or early stage) of recruitment of the adaptive immune system into the infected tissue, such as the lung.

Thus, the pharmaceutical agent is preferably an agent that is suitable for administration to a subject as described above, preferably being suitable for administration at the aforementioned time after the onset of influenza symptoms.

Accordingly, the invention further provides a method of treatment of influenza, comprising administering the pharmaceutical agent of the invention to a patient. Preferably the patient is a patient from one of the groups patients described above.

The invention also provides a method of treatment of influenza, comprising administering the pharmaceutical agent of the invention to a patient at any one or more of the aforementioned points after the onset of influenza symptoms.

The type of influenza referred to in the present invention is not especially limited. Any influenza that can lead to pathological symptoms associated to the over-expression of proinflammatory cytokine may be involved (e.g. influenza A or influenza B). However, preferably the influenza is pandemic influenza or a virulent form of annual influenza. More preferably the influenza is an avian influenza or a porcine influenza.

The present invention further provides a pharmaceutical composition comprising a pharmaceutical agent as defined above and a further additive. The further additive may be any additive commonly added to pharmaceutical agents. Preferably the further additive is an excipient.

The pharmaceutical composition may be adapted for any administration route, but in preferred embodiments it is adapted for parenteral or oral administration. In the most preferred embodiments it is adapted for intravenous administration.

The subject to be treated is not especially limited and may be any vertebrate. Preferably the subject is a mammal or a bird, and more preferably it is a human.

### EXAMPLES

### Example 1

In order to determine the effectiveness of the pharmaceutical agent of the invention, tests were carried out on mice in vivo. A commercial rabbit anti-mouse IFN-γ antibody was obtained for testing (AbD Serotec). The mice were divided into 4 groups, A, B, C and D. Each group comprised 10 mice, five male and five female. Each mouse was challenged intranasally with influenza virus A/PR/8/34 and injected with the pharmaceutical agent 2 days after challenge. Group A received 0.9 μg of isotype control (rabbit IgG). Group B received rabbit anti mouse IFN-γ at a dosing level of 0.9 ug. Group C: received rabbit anti-mouse IFN-γ at a dose of 0.3 μg. Group D received rabbit anti-mouse IFN-γ at a dose of 0.1 μg.

The results of the test, including starting weights and culling weights are shown in Table 1. The survival rate of each group is shown in FIG. 1, whilst the average day-by-day change in weight (as a percentage of starting weight for surviving mice only) is shown in FIG. 2.

TABLE 1

| | | | | | | | culled days |
|---|---|---|---|---|---|---|---|
| | | | | Start | cull | % weight | after |
| Group | Mouse I/D | Sex | Culled | Weight | weight | loss | challenge |
| A | A1 | M | 30/08/2008 | 19.3 | 17.3 | 89.6 | 4 |
| | A2 | M | 30/08/2008 | 19.2 | 15.7 | 81.8 | 4 |
| | A3 | M | 30/08/2008 | 20.2 | 17.4 | 86.1 | 4 |
| | A4 | M | 30/08/2008 | 21.1 | 17.3 | 82.0 | 4 |
| | A5 | M | 30/08/2008 | 21.4 | 17.6 | 82.2 | 4 |
| | A6 | F | 30/08/2008 | 16.1 | 13.0 | 80.7 | 4 |
| | A7 | F | 30/08/2008 | 15.4 | 12.6 | 81.8 | 4 |
| | A8 | F | 30/08/2008 | 14.8 | 12.6 | 85.1 | 4 |
| | A9 | F | 30/08/2008 | 15.1 | 12.9 | 85.4 | 4 |
| | A10 | F | 30/08/2008 | 14.1 | 11.8 | 83.7 | 4 |
| B | B1 | M | 31/08/2008 | 23.5 | 19.1 | 81.3 | 5 |
| | B2 | M | 31/08/2008 | 21.2 | 16.9 | 79.7 | 5 |
| | B3 | M | 08/09/2008 | 20.2 | 19.6 | 97.0 | 12 |
| | B4 | M | 08/09/2008 | 22.1 | 20.9 | 94.6 | 12 |
| | B5 | M | 08/09/2008 | 20.6 | 21.8 | 105.8 | 12 |
| | B6 | F | 30/08/2008 | 13.5 | 11.7 | 86.7 | 4 |
| | B7 | F | 31/08/2008 | 13.4 | 10.9 | 81.3 | 5 |
| | B8 | F | 31/08/2008 | 14.1 | 11.8 | 83.7 | 5 |
| | B9 | F | 31/08/2008 | 14.7 | 12.1 | 82.3 | 5 |
| | B10 | F | 29/08/2008 | 24.2 | 22.8 | 94.2 | 3 |
| C | C1 | M | 08/09/2008 | 20.6 | 21.2 | 102.9 | 12 |
| | C2 | M | 08/09/2008 | 23.5 | 24.1 | 102.6 | 12 |
| | C3 | M | 31/08/2008 | 19.7 | 16.2 | 82.2 | 5 |
| | C4 | M | 30/08/2008 | 21.2 | 18.6 | 87.7 | 4 |
| | C5 | M | 30/08/2008 | 20.7 | 17.9 | 86.5 | 4 |
| | C6 | F | 31/08/2008 | 14.6 | 12.1 | 82.9 | 5 |
| | C7 | F | 30/08/2008 | 14.4 | 12.1 | 84.0 | 4 |
| | C8 | F | 30/08/2008 | 14.7 | 12.7 | 86.4 | 4 |
| | C9 | F | 31/08/2008 | 14.4 | 12.0 | 83.3 | 5 |
| | C10 | F | 08/09/2008 | 14.8 | 14.4 | 97.3 | 8 |
| D | D1 | M | 31/08/2008 | 20.0 | 16.0 | 80.0 | 5 |
| | D2 | M | 08/09/2008 | 19.8 | 21.1 | 106.6 | 12 |
| | D3 | M | 31/08/2008 | 20.5 | 16.8 | 82.0 | 5 |
| | D4 | M | 31/08/2008 | 19.9 | 16.7 | 83.9 | 5 |
| | D5 | M | 29/08/2008 | 21.2 | 18.9 | 89.2 | 3 |
| | D6 | F | 31/08/2008 | 15.6 | 12.8 | 82.1 | 5 |
| | D7 | F | 31/08/2008 | 15.1 | 12.0 | 79.5 | 5 |
| | D8 | F | 30/08/2008 | 15.4 | 12.8 | 83.1 | 4 |
| | D9 | F | 02/09/2008 | 15.6 | 12.8 | 82.1 | 7 |
| | D10 | F | 31/08/2008 | 14.5 | 11.8 | 81.4 | 5 |

The results demonstrate clearly that there is an increased survival rate in mice that have received the pharmaceutical agent, in particular in the higher doses. In the control none of the mice survived, whilst in the higher two doses two and three out of the ten mice survived.

### Example 2

### Mortality and Morbidity Reduction in Mice Lethally Challenged with Influenza A/PR/8/34 Following Treatment with Anti-Cytokine Antibodies

Protocol

Forty-eight (48) C57BL/6 female mice (6-7 weeks old) were divided into four (4) experimental groups containing 12 animals each.

On day 1 animals received an intranasal lethal dose (50 μl total, 25 μl nostril) of influenza A/PR/8/34.

On Day 3 animals received one single intra-peritoneal injection (100 μl) of the following test drugs:
Group A: Monoclonal antibody negative control (61 μg)
Group B: Anti-IFN-γ monoclonal antibody (2.4 μg)
Group C: Anti-TNF-α monoclonal antibody (58 μg)
Group D: Anti-IL-12 monoclonal antibody (9 μg)

All doses tested were intended to reduce, rather than ablate, the cytokine response and hence modulate, rather than completely block, the immune response to the virus.

All animals were assessed daily for morbidity, weight loss and survival until day 6. Morbidity variables (i.e. Body Condition, Posture, Activity, Piloerection, Respiration, Vocalisation, Ataxia and Oculo/Nasal Discharges) were recorded according to the following scale of severity: Normal (0), Mild (1), Laboured (2) and Severe/Cull-point (3).

Statistical differences in weight loss and survival were established using Mann-Whitney non-parametric analysis and Fisher's exact test respectively.

Results

FIG. 3 shows % weight loss for the four groups A, B, C, and D. Weight loss is represented as the average percentage weight of animals within a group on a daily basis compared to the average weight of the same group at the start of the study (day 1).

FIG. 4 shows sum total morbidity for the four groups A, B, C, and D. Sum total morbidity is represented as the sum of all the morbidity scores for all animals still alive within a group at the end of each day.

FIG. 5 shows % survival for the four groups A, B, C, and D. Survival is represented as the percentage animals within a group alive at the end of each day compared to the number of animals that started the study (day 1).

The results show that treatment with anti-TNF-α and anti-IL-12 on day 3 after lethal challenge significantly (p<0.05) improve the survival and reduce the weight loss of infected animals, as well as improving the clinical score and general well being of the animals. It is also important to point out that treatment literally rescues the infected animals from death within 24 hours as clearly shown by the fact that over 80% of control animals succumb to the infection on day 4.

1. A pharmaceutical agent for use in the treatment of inflammation, in a subject prone to and/or experiencing an excessive inflammatory response as a result of infection with an infectious agent and/or exposure to an allergen and/or exposure to an environmental trigger, which pharmaceutical agent comprises an agent for preventing, hindering, modulating or reducing: (a) the production, activity and/or effect of one or more cytokines; and/or (b) the functionality of one or more cells that are targets for the cytokines; and/or (c) a pathological effect caused by cells producing and/or activated by the cytokines.

2. A pharmaceutical agent according to claim 1, wherein the inflammation is acute subacute or chronic inflammation.

3. A pharmaceutical agent according to claim 2, wherein the inflammation is eosinophilic inflammation, or is Delayed Type Hypersensitivity (DTH) inflammation.

4. A pharmaceutical agent according to claim 1, wherein the pathological effect caused by cells producing and/or activated by the cytokines is Acute Respiratory Distress Syndrome (ARDS), Respiratory Distress Syndrome (RDS) or Acute Lung Injury (ALI).

5. A pharmaceutical agent according to claim 1, wherein the infectious agent is selected from an Influenza virus, Haemophilus influenza, SARS virus, Adenovirus, Respiratory Syncitial virus, Streptococcus spp, Staphylococcus spp, Legionella spp, Pseudomonas spp, Klebsiella spp, Burkholderia spp, Pneumococcus spp, Mycobacterium spp, Chlamydia spp, Blastomyces spp, Cryptococcus spp, and Aspergillus spp.

6. A pharmaceutical agent according to claim 1, wherein the allergen is an allergen that causes Asthma.

7. A pharmaceutical agent according to claim 1, wherein the environmental trigger is a trigger that causes Chronic Obstructive Pulmonary Disease (COPD).

8. A pharmaceutical agent according to claim 1, wherein the agent for preventing, hindering, modulating or reducing the production, activity and/or effect of the cytokine is selected from an anti-cytokine antibody and a cytokine-neutralizing fraction of such an antibody.

9. A pharmaceutical agent according to claim 8, wherein the cytokine-neutralizing fraction of the antibody is a F_{ab} fragment.

10. A pharmaceutical agent according to claim 8, wherein the anti-cytokine antibody and/or the cytokine-neutralizing fraction of such an antibody are capable of being delivered as a polypeptide or a recombinant DNA construct.

11. A pharmaceutical agent according to claim 1, wherein the agent for preventing, hindering, modulating or reducing the production, activity and/or effect of the cytokine is a soluble form of a cytokine receptor.

12. A pharmaceutical agent according to claim 11, wherein the agent for preventing, hindering, modulating or reducing the production, activity and/or effect of the cytokine is capable of being delivered as a polypeptide or a recombinant DNA construct.

13. A pharmaceutical agent according to claim 1, wherein the cytokine is selected from a member of the IL-1 superfamily, IL-2, IL-6, IL-8, IL-12, IL-23, alpha, beta and/or gamma interferon, alpha and/or beta TNF, GM-CSF, CCL, CXCL, CX3CL, XCL and/or Lymphotoxin.

14. A pharmaceutical agent according to claim 13, wherein the cytokine is IFN-gamma, TNF-alpha, or IL-12.

15. A pharmaceutical agent according to claim 1, wherein the subject prone to and/or experiencing an excessive inflammatory response to the infection allergen or environmental trigger, shows significantly increased levels of proinflammatory cytokines and/or inflammatory cells in infected or exposed tissue from 48 hours after infection or exposure.

16. A pharmaceutical agent according to claim 15, wherein the tissue comprises lung tissue.

17. A pharmaceutical agent according to claim 1, wherein the subject prone to excessive inflammatory response is a human subject from 13-65 years old.

18. A pharmaceutical agent according to claim 1, wherein the agent is suitable for administration to a subject 48 hours or more after infection or exposure, or at any stage after the onset of pathological symptoms, preferably after the onset of severe pathological symptoms.

19. A pharmaceutical agent according to claim 1, wherein the infectious agent is pandemic influenza.

20. A pharmaceutical agent according to claim 19, wherein the influenza is an avian influenza or a porcine influenza.

21. A pharmaceutical composition comprising
a pharmaceutical agent of claim 1, and
a further additive.

22. A pharmaceutical composition according to claim 21, wherein the further additive is an excipient.

23. A pharmaceutical composition according to claim 21, adapted for parenteral, intranasal or oral administration.

24. A pharmaceutical composition according to claim 21, adapted for intravenous administration.

25. A method of treatment of influenza, comprising administering the pharmaceutical agent of claim 1 or the pharmaceutical composition of claim 21 to a subject.
